(19) United States
(12) Patent Application Publication
(10) Pub. No.: US 2026/0241111 A1
(43) Pub. Date: Aug. 20, 2026

(54) Drug Delivery System With Improved Injection Activation Mechanism

(71) Applicant: Becton, Dickinson and Company, Franklin Lakes, NJ (US)

(72) Inventors: Aditya Vardhan Velagaleti, Secaucus, NJ (US); Craig Schneider, Sparta, NJ (US); Trivikrama Bhanoji Pala Reddy, Parsippany, NJ (US)

(21) Appl. No.: 19/539,261

(22) Filed: Feb. 13, 2026

Related U.S. Application Data
(60) Provisional application No. 63/759,390, filed on Feb. 17, 2025.

(51) Int. Cl. A61M 5/315; A61M 5/20; A61M 5/24; A61M 5/50; A61M 5/31

(52) U.S. Cl. CPC A61M 5/3158; A61M 5/2033; A61M 5/2466; A61M 5/50; A61M 5/206; A61M 2005/2073; A61M 2005/3128; A61M 2209/088

(57) ABSTRACT

Provided herein is a drug delivery system that includes a housing, a needle actuator assembly having a needle and a needle actuator body movable within the housing, and an activation mechanism that interacts with the needle actuator assembly. The activation mechanism includes an actuator button that slides axially from a first position to a second position and an activation wedge affixed to the housing and selectively engageable with a stop wedge of the needle actuator body and a button wedge of the actuator button. With the actuator button in the first position, the activation wedge is engaged with the stop wedge to retain the needle actuator body in a pre-use position and the needle is retracted. With the actuator button slid to the second position, the activation wedge becomes disengaged from the stop wedge, such that the needle actuator body moves to a use position and the needle is extended.

### CROSS-REFERENCE TO RELATED APPLICATION

The present application claims priority to United States Provisional Patent Application No. 63/759,390, entitled “Drug Delivery System With Improved Injection Activation Mechanism” filed February 17, 2025, the entire disclosure of which is hereby incorporated by reference.

### BACKGROUND OF THE INVENTION

### Field of the Invention

The present disclosure relates generally to a drug delivery system and, in particular, to a drug delivery system having an injection activation mechanism that is easily actuatable by a user and resistant to occurrences of unintended activation.

### Description of Related Art

Various types of automatic injection or drug delivery devices have been developed to allow drug solutions and other liquid therapeutic preparations to be administered by untrained personnel or to be self-injected. Generally, these devices include a reservoir that is pre-filled with the liquid therapeutic preparation, and some type of automatic needle-injection mechanism that can be triggered by the user. When the volume of fluid or drug to be administered is generally below a certain volume, such as 1 mL, an auto-injector is typically used, which typically has an injection time of about 10 to 15 seconds. When the volume of fluid or drug to be administered is above 1 mL, the injection time generally becomes longer resulting in difficulties for the patient to maintain contact between the device and the target area of the patient’s skin. Further, as the volume of drug to be administered becomes larger, increasing the time period for injection becomes desirable. The traditional method for a drug to be injected slowly into a patient is to initiate an IV and inject the drug into the patient’s body slowly. Such a procedure is typically performed in a hospital or outpatient setting.

Certain devices allow for self-injection in a home setting and are capable of gradually injecting a liquid therapeutic preparation into the skin of a patient. In some cases, these devices are small enough (both in height and in overall size) to allow them to be “worn” by a patient while the liquid therapeutic preparation is being infused into the patient. These devices typically include a drive assembly or discharge mechanism to force the liquid therapeutic preparation to flow out of a container and into the injection needle. Such devices also typically include a valve or flow control mechanism to cause the liquid therapeutic preparation to begin to flow at the proper time and an activation mechanism (e.g., actuator button) to initiate the injection.

In existing wearable injectors as described above, the injector is configured to operate through the engagement of an actuator button by a user, which results in: actuating a needle actuator assembly to cause a needle thereof to pierce the skin of a user, actuating a drive assembly to place the needle in fluid communication with the container and to expel fluid or medicament from the container, and the withdrawing the needle after injection of the medicament is complete.

The activation mechanism in existing wearable injectors includes an interaction between the activation mechanism and needle actuator that causes the needle actuator to tilt and drive forward. While this tilting and driving interaction between the activation mechanism and needle actuator is effective in actuating the needle from its retracted position to its extended position, it is recognized that there are several drawbacks to the design. As one example, the tilting and driving interaction between the activation mechanism and needle actuator may require a user to push the actuator button of the injector with a large amount of force in order to initiate the activation, with this force becoming higher in worst-case tolerance conditions. As another example, the tilting and driving interaction between the activation mechanism and needle actuator can lead to an unintended activation of the needle actuator, with it recognized that a dropping of the wearable injector (onto the floor, for example) may initiate activation of the needle actuator and cause the needle to prematurely move from its retracted position to its extended position.

Accordingly, a need exists for a drug delivery system, such as a wearable injector, that includes an improved design regarding the activation mechanism and interaction thereof with the needle assembly. The activation mechanism would be easily actuatable by a user, but also resistant to occurrences of unintended activation, such as when the injector is dropped by a user.

### SUMMARY OF THE INVENTION

Provided herein is a drug delivery system that includes a housing and a needle actuator assembly comprising a needle having a retracted position where the needle is positioned within the housing and an extended position where a portion of the needle protrudes from the housing, a needle actuator body movable within the housing and including a stop wedge, the needle actuator body movable from a pre-use position where the needle is in the retracted position to a use position where the needle is in the extended position, and a needle shuttle that secures the needle and is received within the needle actuator body, the needle shuttle configured to engage the needle actuator body as it moves from the pre-use position to the use position to move the needle from the retracted position and the extended position. The drug delivery system also includes an activation mechanism that interacts with the needle actuator assembly, with the activation mechanism comprising an actuator button comprising a user interface portion and a button wedge, the actuator button configured to slide axially relative to the housing from a first position to a second position, and an activation wedge affixed to the housing and comprising a first engagement member engageable with the stop wedge and a second engagement member engageable with the button wedge, with the first engagement member engaged with the stop wedge when the actuator button is in the first position to retain the needle actuator body in the pre-use position. As the actuator button is slid from the first position toward the second position, the button wedge engages the second engagement member, forcing the activation wedge to move and the first engagement member to become disengaged from the stop wedge, thereby releasing the needle actuator body such that the needle actuator body moves from the pre-use position to the use position and the needle moves from the retracted position to the extended position.

In some embodiments, the drug delivery system further includes a container received within the housing, the container configured to receive a medicament and comprising a stopper configured to move within the container, a valve assembly configured to pierce a closure of the container and place the medicament within the container in fluid communication with the needle, and a drive assembly configured to move the container toward the valve assembly and to move the stopper within the container to deliver the medicament to the needle, wherein the container is spaced from the valve assembly when the actuator button is in the first position, and wherein the container is engaged with the valve assembly when the actuator button is moved toward the second position.

In some embodiments, the button wedge comprises a sloped bottom surface and the second engagement member comprises a sloped top surface, and wherein as the actuator button is slid from the first position toward the second position, the sloped bottom surface slides along the sloped top surface and forces the activation wedge to move.

In some embodiments, the activation wedge comprises a support leg joined to the housing at one end thereof and to a main body of the activation wedge at another end thereof, the first engagement member provided on the main body and the second engagement member joined to the main body, wherein the support leg comprises a spring-type member that actuates between a relaxed configuration and a compressed configuration to vary a length of the support leg.

In some embodiments, sliding of the actuator button from the first position toward the second position applies a downward directed force to the activation wedge, causing the support leg to transition from the relaxed configuration to the compressed configuration, and thereby moving the first engagement member and the second engagement member vertically downward.

In some embodiments, the first engagement member comprises a vertically-oriented, rearward-facing surface and the stop wedge comprises a vertically-oriented, forward-facing surface, with the rearward-facing surface sliding downward and out of engagement with the forward-facing surface as the support leg transition to the compressed configuration, such that the first engagement member becomes disengaged from the stop wedge.

In some embodiments, a vertical distance by which the support leg compresses is greater than a vertical height of the forward-facing surface of the stop wedge.

In some embodiments, the stop wedge protrudes radially outward from a remainder of the needle actuator body and the first engagement member protrudes radially inward from a remainder of the main body of the activation wedge.

In some embodiments, the needle actuator body comprises guide surfaces formed on a radially inward-facing surface thereof and the needle shuttle comprises cam surfaces thereon, with the needle shuttle and the needle configured to move between the retracted position and the extended position through engagement between the guide surfaces and the cam surfaces.

In some embodiments, the drug delivery system includes an extension spring that biases the needle actuator body from the pre-use position to the use position.

In some embodiments, in sliding axially relative to the housing, the actuator button moves only in a horizontal direction, without any tilting of the actuator button.

In some embodiments, the needle actuator body is movable to a post-use position, and wherein the actuator button comprises lockout arms that engage a portion of the needle actuator body as the needle actuator body moves to the post-use position, thereby preventing further movement of the actuator button.

In some embodiments, the actuator button comprises a central arm and retention arms that engage the housing and the needle actuator body, respectively, during movement of the actuator button between the first position and the second position and during movement of the needle actuator body from the pre-use position, to the use position, and to the post-use position.

In some embodiments, the drug delivery system includes a pad configured to seal the needle, wherein the pad is biased into the needle as the needle actuator body moves from the use position to the post-use position.

In some embodiments, the drug delivery system comprises a wearable injector.

Also provided herein is a drug delivery system that includes a housing and a needle actuator assembly comprising a needle having a retracted position where the needle is positioned within the housing and an extended position where a portion of the needle protrudes from the housing and a needle actuator body movable within the housing and being movable from a pre-use position where the needle is in the retracted position to a use position where the needle is in the extended position. The drug delivery system also includes an activation mechanism that interacts with the needle actuator assembly, the activation mechanism comprising an actuator button configured to slide axially relative to the housing from a first position to a second position and an activation wedge affixed to the housing and comprising a spring-type support leg actuatable between a relaxed configuration and a compressed configuration, with the activation wedge engaged with the needle actuator body when the support leg is in the relaxed configuration and disengaged from the needle actuator body when the support leg is in the compressed configuration. As the actuator button is slid axially from the first position toward the second position, the actuator button engages the activation wedge and actuates the spring-type support leg from the relaxed configuration to the compressed configuration, such that the activation wedge vertically disengages the needle actuator body, allowing the needle actuator body to move from the pre-use position to the use position.

In some embodiments, the needle actuator body comprises a stop wedge, the actuator button comprises a button wedge, and the activation wedge comprises a first engagement member engageable with the stop wedge and a second engagement member engageable with the button wedge, with the first engagement member engaged with the stop wedge when the actuator button is in the first position to retain the needle actuator body in the pre-use position, wherein as the actuator button is slid from the first position toward the second position, the button wedge engages the second engagement member, forcing the activation wedge to move and the first engagement member to become disengaged from the stop wedge, thereby releasing the needle actuator body such that the needle actuator body moves from the pre-use position to the use position and the needle moves from the retracted position to the extended position.

In some embodiments, the button wedge comprises a sloped bottom surface and the second engagement member comprises a sloped top surface, and wherein as the actuator button is slid from the first position toward the second position, the sloped bottom surface slides along the sloped top surface and applies a downward force to the activation wedge that actuates the spring-type support leg from the relaxed configuration to the compressed configuration, such that the activation wedge is moved downward and vertically disengages the needle actuator body.

In some embodiments, the drug delivery system includes an extension spring that biases the needle actuator body from the pre-use position to the use position.

In some embodiments, the needle actuator assembly comprises a needle shuttle that secures the needle and is received within the needle actuator body, the needle shuttle configured to engage the needle actuator body as it moves from the pre-use position to the use position to move the needle from the retracted position and the extended position.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a drug delivery system according to one aspect of the present invention.

FIG. 2 is a perspective, cross-sectional view of the drug delivery system of FIG. 1 according to one aspect of the present invention.

FIG. 3 is a front, cross-sectional view of the drug delivery system of FIG. 1 according to one aspect of the present invention.

FIG. 4 is a top view of the drug delivery system of FIG. 1 according to one aspect of the present invention, showing a top portion of the housing removed and the drug delivery system in a pre-use position.

FIG. 5 is a top, cross-sectional view of the drug delivery system of FIG. 1 according to one aspect of the present invention, showing the drug delivery system in a pre-use position.

FIG. 6 is a front, cross-sectional view of the drug delivery system of FIG. 1 according to one aspect of the present invention, showing the drug delivery system in a pre-use position.

FIG. 7 is a top view of the drug delivery system of FIG. 1 according to one aspect of the present invention, showing a top portion of the housing removed and the drug delivery system in an initial actuation position.

FIG. 8 is a top, cross-sectional view of the drug delivery system of FIG. 1 according to one aspect of the present invention, showing the drug delivery system in an initial actuation position.

FIG. 9 is a front, cross-sectional view of the drug delivery system of FIG. 1 according to one aspect of the present invention, showing the drug delivery system in an initial actuation position.

FIG. 10 is a top view of the drug delivery system of FIG. 1 according to one aspect of the present invention, showing a top portion of the housing removed and the drug delivery system in a use position.

FIG. 11 is a top, cross-sectional view of the drug delivery system of FIG. 1 according to one aspect of the present invention, showing the drug delivery system in a use position.

FIG. 12 is a front, cross-sectional view of the drug delivery system of FIG. 1 according to one aspect of the present invention, showing the drug delivery system in a use position.

FIG. 13 is a top view of the drug delivery system of FIG. 1 according to one aspect of the present invention, showing a top portion of the housing removed and the drug delivery system in a post-use position.

FIG. 14 is a top, cross-sectional view of the drug delivery system of FIG. 1 according to one aspect of the present invention, showing the drug delivery system in a post-use position.

FIG. 15 is a front, cross-sectional view of the drug delivery system of FIG. 1 according to one aspect of the present invention, showing the drug delivery system in a post-use position.

FIG. 16 is a perspective, cross-sectional view of the drug delivery system of FIG. 1 according to one aspect of the present invention, showing a pad with the drug delivery system in a pre-use position.

FIG. 17 is a front view of a needle actuator assembly that may be included in the drug delivery system of FIG. 1 according to one aspect of the present invention.

FIG. 18 is a left-side perspective view of a needle shuttle of the needle actuator assembly of FIG. 17 according to one aspect of the present invention.

FIG. 19 is a right-side perspective view of a needle shuttle of the needle actuator assembly of FIG. 17 according to one aspect of the present invention.

FIG. 20A is a front view of the needle actuator assembly of FIG. 17 according to one aspect of the present invention, showing the needle actuator assembly in a pre-use position.

FIG. 20B is a front view of the needle actuator assembly of FIG. 17 according to one aspect of the present invention, showing the needle actuator assembly in a use position.

FIG. 20C is a front view of the needle actuator assembly of FIG. 17 according to one aspect of the present invention, showing the needle actuator assembly in an initial post-use position.

FIG. 20D is a front view of the needle actuator assembly of FIG. 17 according to one aspect of the present invention, showing the needle actuator assembly in a post-use position.

FIG. 21 is a perspective view of an actuator button that may be used in combination with the needle actuator assembly of FIG. 17 according to a further aspect of the present invention.

FIG. 22 is a bottom view of the actuator button of FIG. 21 according to a further aspect of the present invention.

FIG. 23A is a top view of the actuator button of FIG. 21 relative to a top portion of the housing according to a further aspect of the present invention, showing the actuator button in a pre-use position.

FIG. 23B is a top view of the actuator button of FIG. 21 relative to a top portion of the housing according to a further aspect of the present invention, showing the actuator button in a use position.

FIG. 24A is a perspective, cross-sectional view of an activation mechanism for actuating the needle actuator assembly of FIG. 17 according to one aspect of the present invention, showing the activation mechanism and the needle actuator assembly in a pre-use position.

FIG. 24B is a perspective, cross-sectional view of an activation mechanism for actuating the needle actuator assembly of FIG. 17 according to one aspect of the present invention, showing the activation mechanism and the needle actuator assembly in a use position.

### DETAILED DESCRIPTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

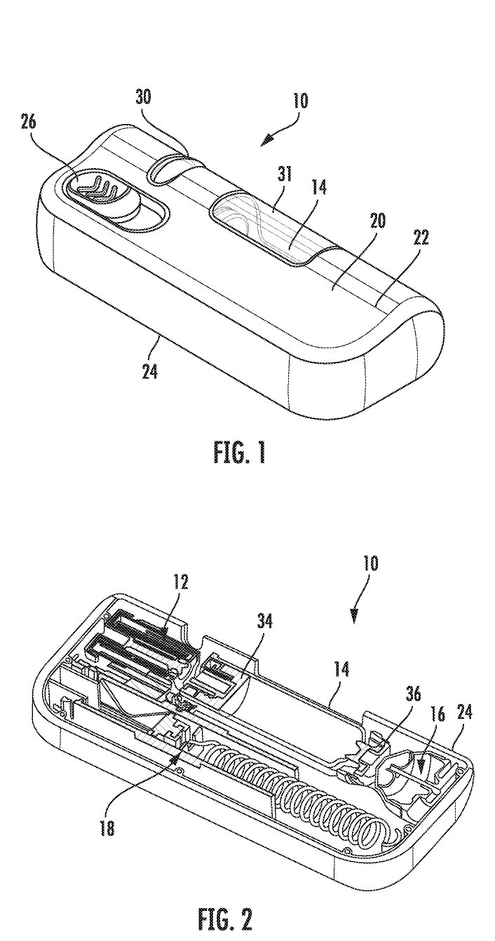

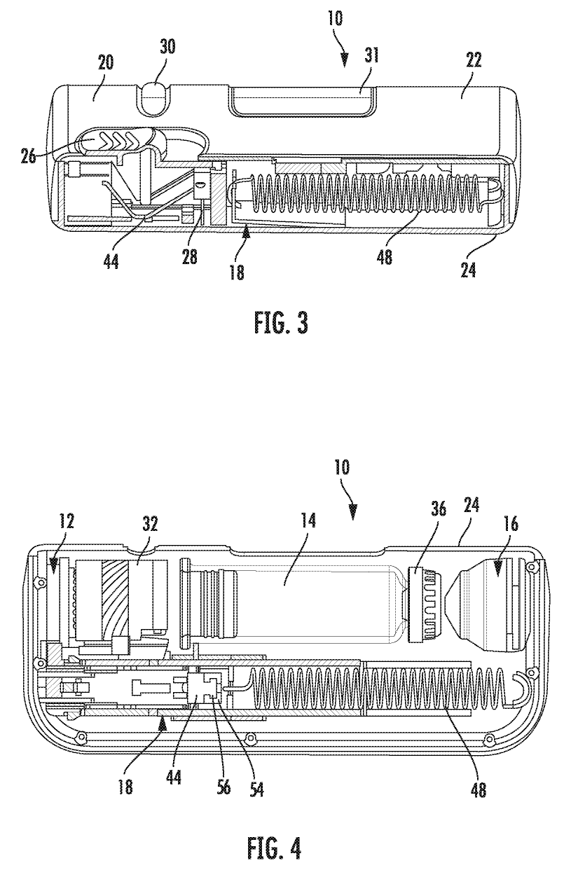

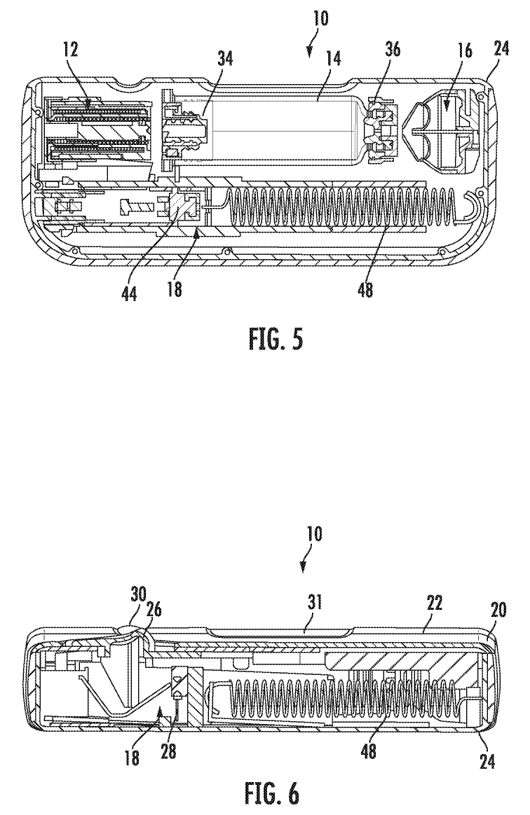

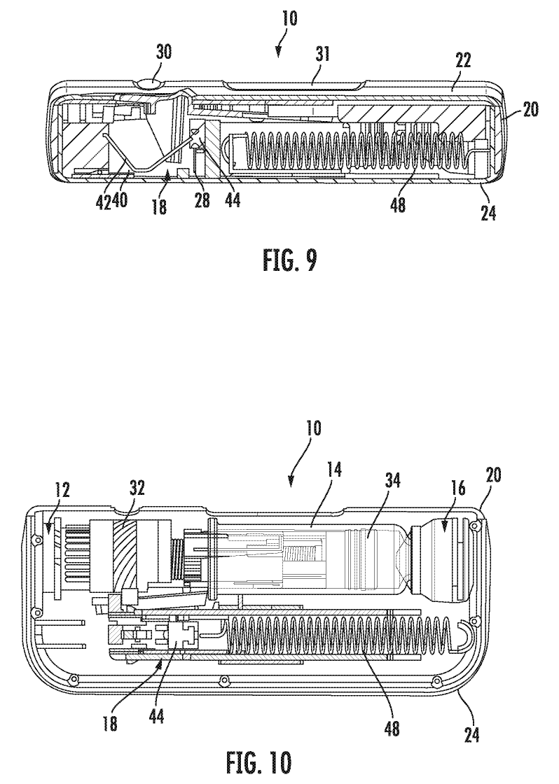

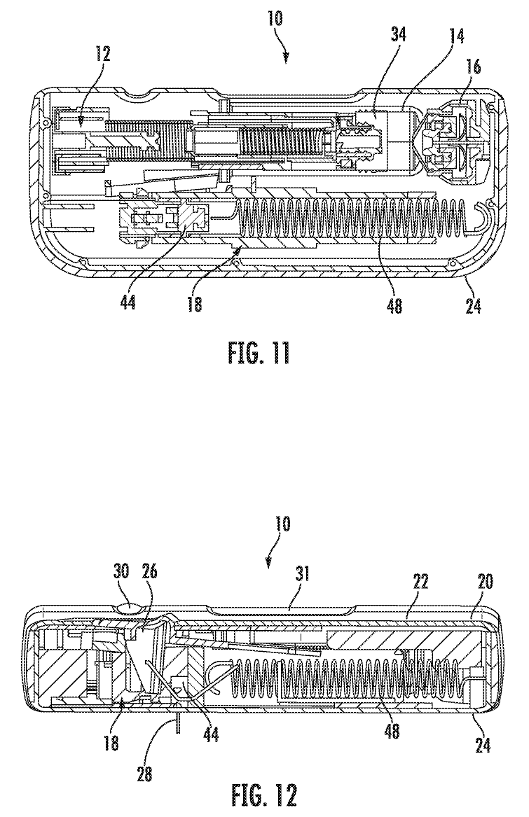

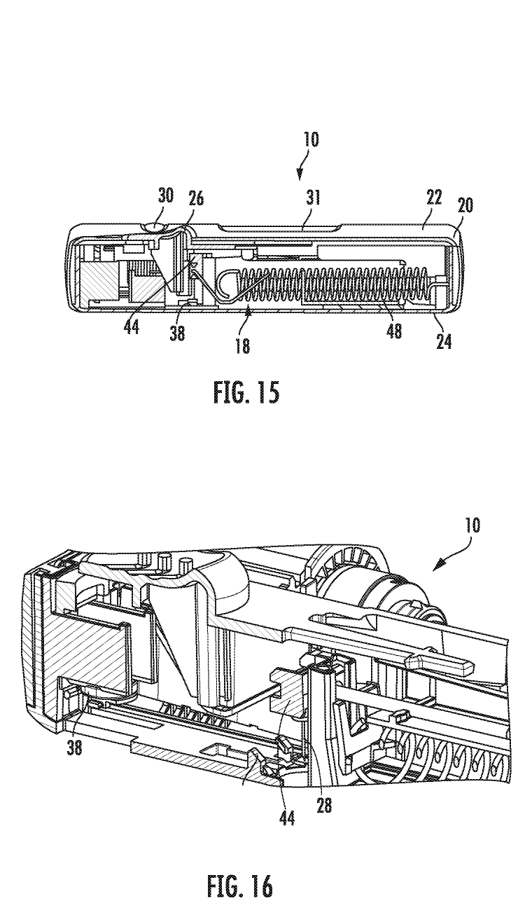

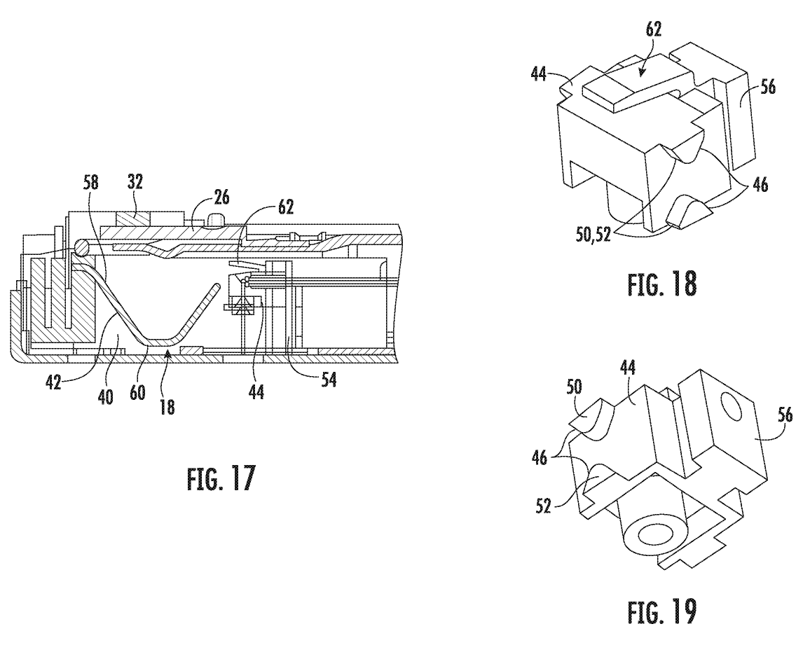

Referring to FIGS. 1-16, a drug delivery system 10 according to one aspect of the present invention includes a drive assembly 12, a container 14, a valve assembly 16, and a needle actuator assembly 18. The drive assembly 12, the container 14, the valve assembly 16, and the needle actuator assembly 18 are at least partially positioned within a housing 20. The housing 20 includes a top portion 22 and a bottom portion 24, although other suitable arrangements for the housing 20 may be utilized. In one aspect, the drug delivery system 10 is an injector device configured to be worn or secured to a user and to deliver a predetermined dose of a medicament provided within the container 14 via injection into the user. The system 10 may be utilized to deliver a “bolus injection” where a medicament is delivered within a set time period. The medicament may be delivered over a time period of up to 45 minutes, although other suitable injection amounts and durations may be utilized. A bolus administration or delivery can be carried out with rate controlling or have no specific rate controlling. The system 10 may deliver the medicament at a fixed pressure to the user with the rate being variable. The general operation of the system 10 is described below in reference to FIGS. 1-16 with the specifics of the needle actuator assembly 18 and other features of the system 10 discussed below in connection with FIGS. 17-24.

Referring again to FIGS. 1-16, the system 10 is configured to operate through the engagement of an actuator button 26 by a user, which results in a needle 28 of the needle assembly 18 piercing the skin of a user, the actuation of the drive assembly 12 to place the needle 28 in fluid communication with the container 14 and to expel fluid or medicament from the container 14, and the withdrawal of the needle 28 after injection of the medicament is complete. The general operation of a drug delivery system is shown and described in U.S. Patent No. 10,549,044 to Becton Dickinson and Company, which is hereby incorporated by reference in its entirety. The housing 20 of the system 10 includes an indicator window 30 for viewing an indicator arrangement 32 configured to provide an indication to a user on the status of the system 10 and a container window 31 for viewing the container 14. The indicator window 30 may be a magnifying lens for providing a clear view of the indicator arrangement 32. The indicator arrangement 32 moves along with the needle actuator assembly 18 during use of the system 10 to indicate a pre-use status, use status, and post-use status of the system 10. The indicator arrangement 32 provides visual indicia regarding the status, although other suitable indicia, such an auditory or tactile, may be provided as an alternative or additional indicia.

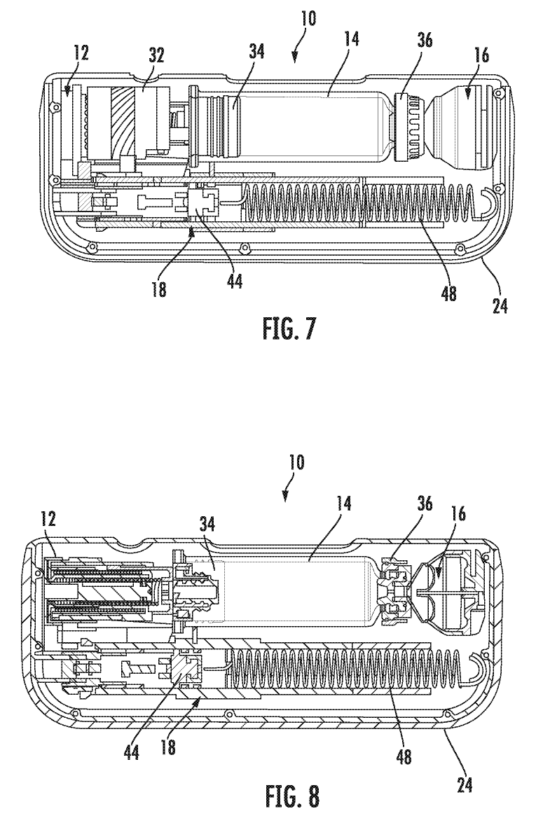

Referring to FIGS. 4-6, during a pre-use position of the system 10, the container 14 is spaced from the drive assembly 12 and the valve assembly 16 and the needle 28 is in a retracted position. During the initial actuation of the system 10, as shown in FIGS. 7-9, the drive assembly 12 engages the container 14 to move the container 14 toward the valve assembly 16, which is configured to pierce a closure 36 of the container 14 and place the medicament within the container 14 in fluid communication with the needle 28 via a tube (not shown) or other suitable arrangement. The drive assembly 12 is configured to engage a stopper 34 of the container 14, which will initially move the entire container 14 into engagement with the valve assembly 16 due to the incompressibility of the fluid or medicament within the container 14. The initial actuation of the system 10 is caused by engagement of the actuator button 26 by a user, which releases the needle actuator assembly 18 and the drive assembly 12 as discussed below in more detail. During the initial actuation, the needle 28 is still in the retracted position and about to move to the extended position to inject the user of the system 10.

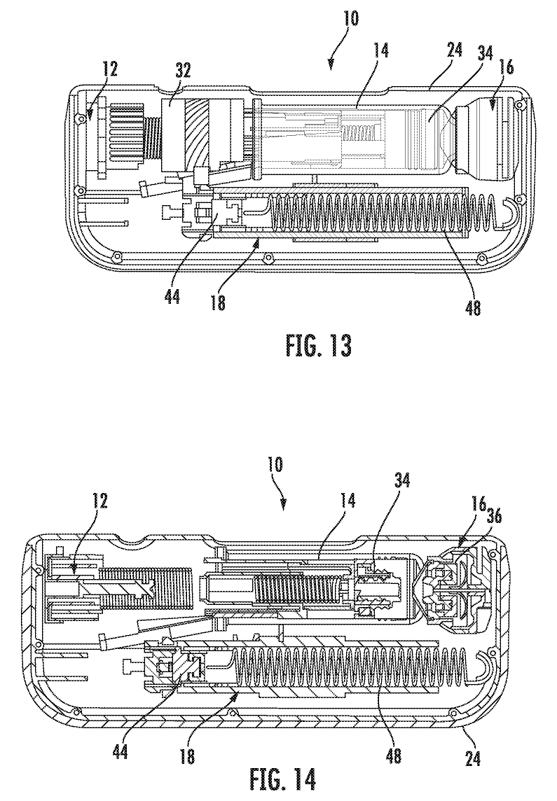

During the use position of the system 10, as shown in FIGS. 10-12, the needle 28 is in the extended position at least partially outside of the housing 20 with the drive assembly 12 moving the stopper 34 within the container 14 to deliver the medicament from the container 14, through the needle 28, and to the user. In the use position, the valve assembly 16 has already pierced a closure 36 of the container 14 to place the container 14 in fluid communication with the needle 28, which also allows the drive assembly 12 to move the stopper 34 relative to the container 14 since fluid is able to be dispensed from the container 14. At the post-use position of the system 10, shown in FIGS. 13-15, the needle 28 is in the retracted position and engaged with a pad 38 to seal the needle 28 and prevent any residual flow of fluid or medicament from the container 14. The container 14 and valve assembly 16 may be the container 14 and valve assembly 16 shown and described in International Publication No. WO 2015/081337, which is hereby incorporated by reference in its entirety.

Referring to FIGS. 1-16 and 17-20D, a needle actuator assembly 18 according to one aspect of the disclosure is shown. The needle actuator assembly 18 includes a needle actuator body 40 having guide surfaces 42, a needle shuttle 44 having cam surfaces 46, and the needle 28 received by the needle shuttle 44 and configured to be in fluid communication with the container 14 as discussed above. The needle actuator body 40 is generally rectangular with the guide surfaces 42 protruding radially inward. The needle shuttle 44 is received within the needle actuator body 40. The needle actuator body 40 is moveable within the housing 20 from a pre-use position (shown in FIGS. 4-6), an initial actuation position (FIGS. 7-9), a use position (FIGS. 10-12), and a post-use position (FIGS. 13-15). The needle actuator body 40 is biased from the pre-use position to the post-use position via an extension spring 48, although other suitable biasing arrangements may be utilized. The needle actuator body 40 may be released and free to move from the pre-use position to the use position upon engagement of the actuator button 26, which is discussed in more detail below.

Referring to FIGS. 16-20D, the needle shuttle 44 is moveable along a vertical axis between a retracted position where the needle 28 is positioned within the housing 20 and an extended position where at least a portion of the needle 28 extends out of the housing 20. The needle shuttle 44 is configured to move between the retracted position and the extended position through engagement between the guide surfaces 42 of the needle actuator body 40 and the cam surfaces 46 of the needle shuttle 44. The cam surfaces 46 are provided by first and second cam members 50, 52, with the first cam member 50 spaced from the second cam member 52. The housing 20 includes a guide post 54 having a recess configured to receive a T-shaped projection 56 on the needle shuttle 44, although other shapes and configurations may be utilized for the guide post 54 and T-shaped projection 56. The needle shuttle 44 moves along the guide post 54 between the retracted and extended positions. The guide post 54 is linear and extends about perpendicular from the housing 20, although other suitable arrangements may be utilized. The guide surfaces 42 of the needle actuator body 40 are non-linear and each include a first side 58 and a second side 60 positioned opposite from the first side 58.

As discussed below, the guide surfaces 42 of the needle actuator body 40 cooperate with the cam members 50, 52 of the needle shuttle 44 to move the needle shuttle 44 vertically between the retracted and extended positions as the needle actuator body 40 moves axially from the pre-use position to the post-use position. The needle shuttle 44 also includes a shuttle biasing member 62 configured to engage the housing 20 or the actuator button 26. In particular, the shuttle biasing member 62 engages the housing 20 or actuator button 26 and provides a biasing force when the needle actuator body 40 is transitioning from the use position to the post-use position. When the needle actuator body 40 is fully transitioned to the post-use position, the cam members 50, 52 of the needle shuttle 44 are disengaged from the guide surfaces 42 of the needle actuator body 40 and the shuttle biasing member 62 biases the needle shuttle 44 downward such that the needle 28 engages the pad 38, as discussed above. As discussed above in connection with FIGS. 1-16, however, the pad 38 may also be biased into the needle 28 rather than biasing the needle shuttle 44 downwards via the shuttle biasing member 62.

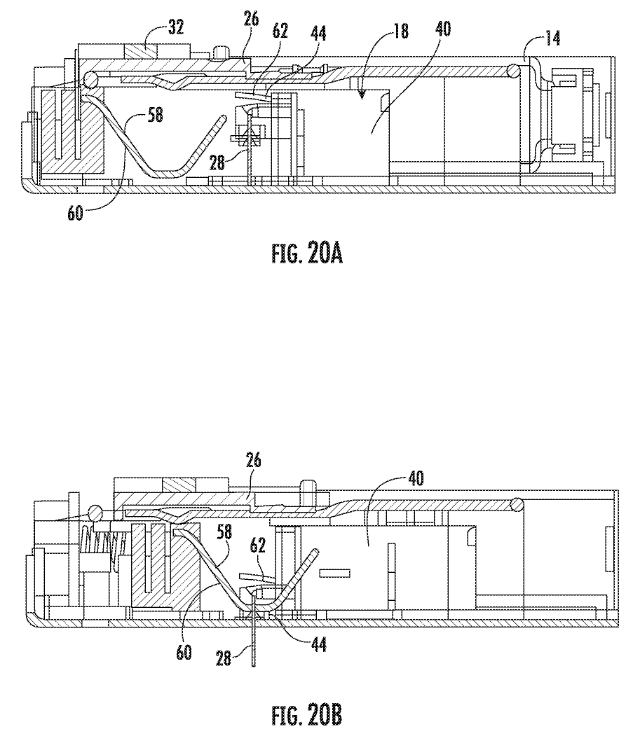

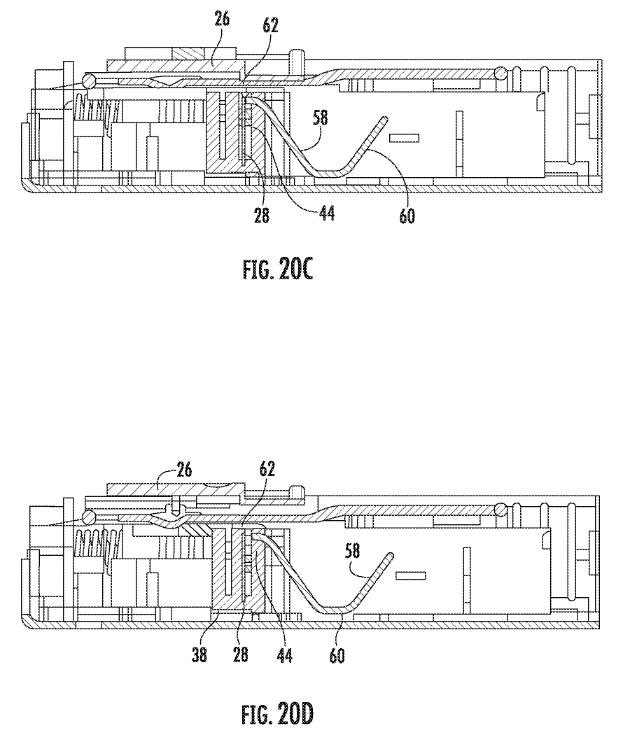

Referring to FIGS. 20A-20D, in a pre-use position (FIG. 20A), the needle shuttle 44 is in the retracted position with the cam members 50, 52 spaced from the guide surfaces 42 of the needle actuator body 40. As the needle actuator body 40 moves to the use position (FIG. 20B), the second cam member 52 of the needle shuttle 44 engages the second side 60 of the guide surfaces 42 to move the needle shuttle 44 from the retracted position to the extended position. During the transition from the use position to the post-use position of the needle actuator body 40 (FIG. 20C), the first cam member 50 of the needle shuttle 44 is engaged with the first side 58 of the guide surfaces 42 to move the needle shuttle 44 from the second position to the first position. After the needle actuator body 40 is fully transitioned to the post-use position (FIG. 20D), the shuttle biasing member 62 biases the needle shuttle 44 downward as the cam members 50, 52 disengage from the guide surfaces 42 of the needle actuator body 40 with the needle 28 engaging the pad 38. The transition of the needle actuator body 40 and the corresponding position of the needle shuttle 44 is also shown in FIGS. 20A-20D.

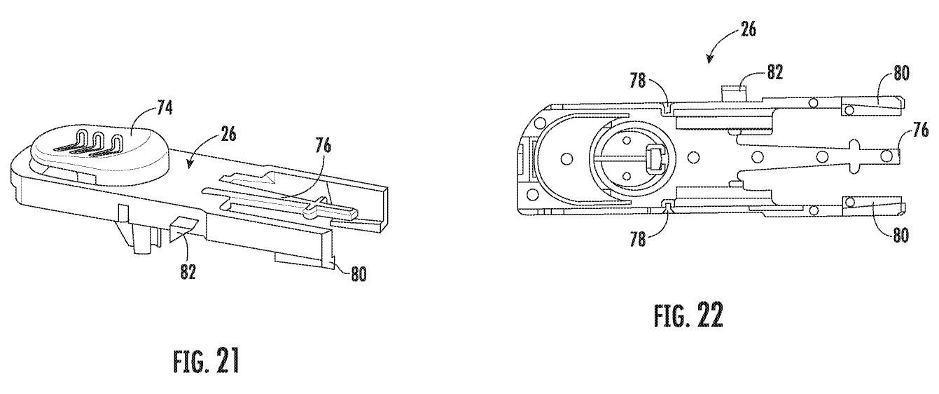

Referring to FIGS. 21-24B, an activation mechanism 70 for interacting with and actuating the needle actuator assembly 18 is illustrated according to one aspect of the present invention is shown. The activation mechanism 70 may be considered to include the actuator button 26 and an activation wedge 72 that may be formed as part of housing 20. The activation mechanism 70 is configured to provide one or more of the following features, which are discussed in more detail below: one-way axial displacement or sliding of the actuator button 26 and lockout of the actuator button 26 in the post-use position of the needle actuator body 40.

As best shown in FIGS. 21 and 22, the actuator button 26 may comprise a single, integrally formed component that includes a user interface portion 74 for interacting with a user, a central arm 76, lockout arms 78, retention arms 80, and a button wedge 82.

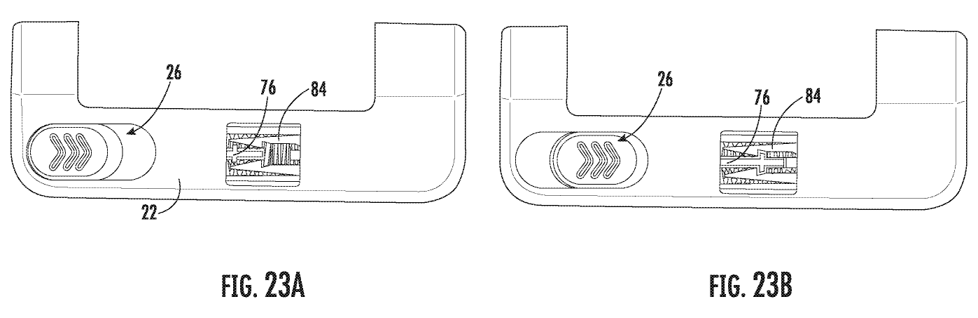

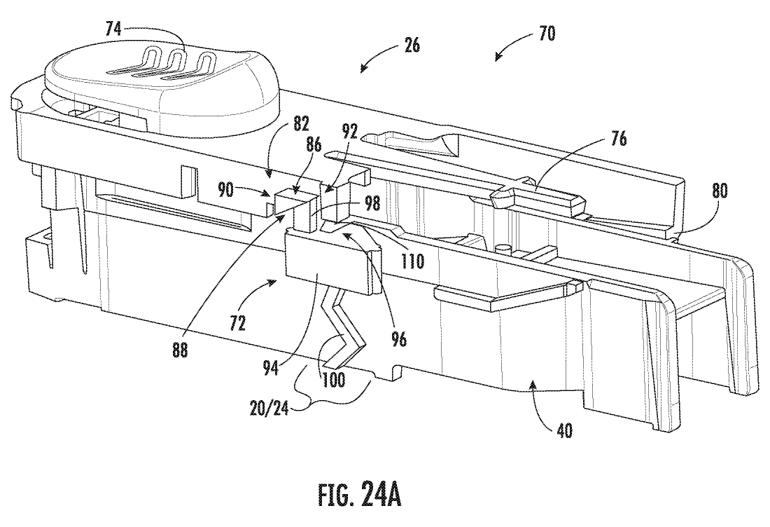

As best shown in FIGS. 23A and 23B, the central arm 76 engages prongs 84 in the top portion 22 of the housing 20. During transition of the system 10 from the pre-use position (FIG. 23A) to the use position (FIG. 23B), the central arm 76 slides axially relative to prongs 84 to ensure alignment and axial sliding of the actuator button 26 relative to housing 20 and needle actuator body 40.

During transition between the pre-use position and the use position, the retention arms 80 engage a portion of the needle actuator body 40 to maintain alignment between the actuator button 26 and the needle actuator body 40 until the post-use position is reached.

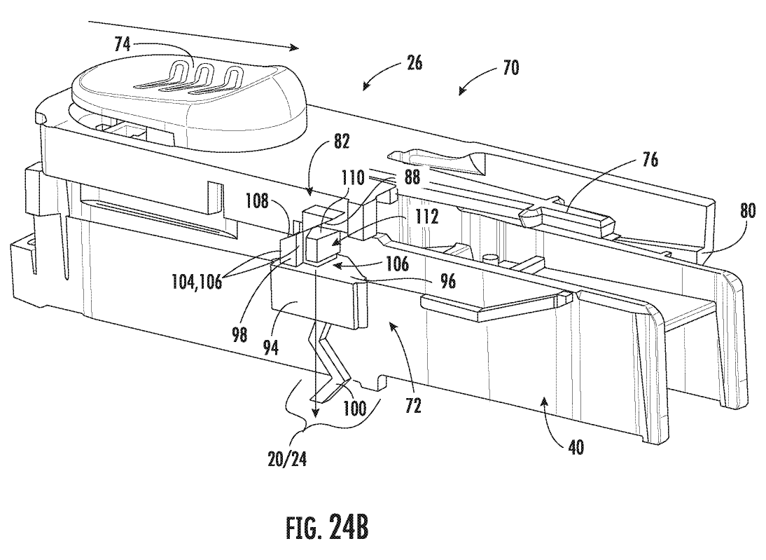

As shown in FIGS. 24A and 24B, the button wedge 82 may comprise a wedge-shaped member that extends radially outward from a main body portion of actuator button 26, with the button wedge 82 positioned lengthwise on the actuator button 26 at a location between user interface portion 74 and retention arms 80. The button wedge 82 may comprise a generally flat top surface 86 and a sloped bottom surface 88, with the sloped bottom surface 88 sloping upward in a direction from back to front – such that a back edge/surface 90 of the button wedge 82 is a vertically oriented surface and the sloped bottom surface 88 slopes upward to a point at a front edge 92 of the button wedge 82. During transition between the pre-use position and the use position, the button wedge 82 engages a portion of the activation wedge 72, with the activation wedge 72 in-turn engaging a portion of the needle actuator body 40, to selectively allow the needle actuator body 40 to move from the pre-use position to the use position, as explained in further detail below.

The activation wedge 72 is formed on housing 20 at a location that provides for interaction of the activation wedge 72 with each of the actuator button 26 and needle actuator body 40. The activation wedge 72 is configured as a generally vertically-oriented member that includes a main body 94 having an actuation body engagement member 96 (hereafter, a “first engagement member 96”), an actuator button engagement member 98 (hereafter, a “second engagement member 98”), and a support leg 100 – with the second engagement member 98 extending upward from the main body 94 and the support leg 100 extending downward from the main body 94.

The first engagement member 96 of main body 94 is provided as a body segment that protrudes radially inward from a remainder of the main body 94. The first engagement member 96 presents a rearward-facing surface 102 that is flat and generally orthogonal to the direction of travel of the needle actuator body 40.

The second engagement member 98 of activation wedge 72 is positioned and configured to engage and interact with the button wedge 82 of actuator button 26. The second engagement member 98 may comprise vertically oriented back and front surfaces 104, 106 and a sloped top surface 108 extending between the back and front surfaces 104, 106. The sloped top surface 108 slopes upward in a direction from back to front – with the sloped top surface 108 of second engagement member 98 thus arranged/configured to interact with the sloped bottom surface 88 of button wedge 82 on actuator button 26. During transition between the pre-use position and the use position, the second engagement member 98 of activation wedge 72 may thus engage the button wedge 82, as explained in further detail below.

The support leg 100 extends downwardly from the main body 94 and functions to secure the activation wedge 72 to the housing 20. In some embodiments, the activation wedge 72 may be formed integrally with housing 20 (i.e., with bottom portion 24), such that a bottom end of support leg 100 is integrally joined to the housing 20. In other embodiments, a bottom end of support leg 100 may be inter-fit with a receptacle of the housing 20, to secure the activation wedge 72 relative to the housing 20. The support leg 100 is configured to have spring-type properties that provides for compression and extension thereof – so as to alter/change a length of the support leg 100. The support leg 100 may thus be formed of a flexible/bendable material and have a shape suitable for deflecting, such a zig-zag or spring-shaped structure. Compression of the support leg 100 thus allows for a positioning of the activation wedge 72 to change relative to the actuator button 26 and the needle actuator body 40, as occurs during transition between the pre-use position and the use position, as explained in further detail below.

As indicated above, the needle actuator body 40 engages and interacts with activation wedge 72 to selectively allow the needle actuator body 40 to move from the pre-use position to the use position. In particular, needle actuator body 40 includes a stop wedge 110 formed thereon that interacts with the first engagement member 96 of activation wedge 72. The stop wedge 110 protrudes radially outward from a remainder of the needle actuator body 40. The stop wedge 110 presents a forward-facing surface 112 that is flat and generally orthogonal to the direction of travel of the needle actuator body 40. The stop wedge 110 is formed on needle actuator body 40 so as to be vertically aligned with the main body 94 of activation wedge 72, when the support leg 100 of activation wedge 72 is a relaxed/uncompressed configuration, such that the forward-facing surface 112 of stop wedge 110 is aligned with and engages the rearward-facing surface 102 of first engagement member 96. With the stop wedge 110 engaged with the first engagement member 96 of activation wedge 72 in this manner, a distal axial movement of the needle actuator body 40 is prevented – with the needle actuator body 40 being held in a pre-use position.

To actuate the system 10 using the actuator button 26, the user first slides the user interface portion 74 in a first axial direction, shown as being to the right in As shown in FIGS. 24A and 24B. The user may be required to slide the user interface portion 74 about 10 mm or about 8 mm, although other suitable distances may be utilized. Moving the actuator button 26 axially moves the button wedge 82 of actuator button 26 into contact and engagement with activation wedge 72. More specifically, sloped bottom surface 88 of button wedge 82 engages the sloped top surface 108 of second engagement member 98 of activation wedge 72, as shown in FIG. 24A. As the user continues to slide the user interface portion 74 in the first axial direction, the sloped bottom surface 88 of button wedge 82 is pressed into the sloped top surface 108 of second engagement member 98, which forces the activation wedge 72 downward, such that the support leg 100 transitions from a relaxed/uncompressed configuration to a compressed configuration.

As the support leg 100 of activation wedge 72 transitions from its relaxed/uncompressed configuration to its compressed configuration, the vertical positioning of the activation wedge 72 is changed, with both the main body 94 (and first engagement member 96 thereof) and the second engagement member 98 shifting down, as shown in FIG. 24B. As the first engagement member 96 of the activation wedge 72 moves down, it slides out of contact with the stop wedge 110 formed on needle actuator body 40 – i.e., the rearward-facing surface 102 of first engagement member 96 becomes vertically misaligned with the forward-facing surface 112 of stop wedge 110 – until the first engagement member 96 is no longer in contact with the stop wedge 110. According to embodiments of the disclosure, a vertical distance by which the support leg 100 compresses is greater than a vertical height of the forward-facing surface 112 of the stop wedge 110, so as to allow for the first engagement member 96 to disengage from the stop wedge 110. The disengaging of the first engagement member 96 from the stop wedge 110 releases the needle actuator body 40 and allows a distal axial movement thereof, thereby transitioning the needle actuator body 40 from the pre-use position (FIG. 24A) to the use position (FIG. 24B).

With the needle actuator body 40 disengaged from the activation wedge 72, the needle actuator body 40 continues to move from the use position to the post-use position, as shown in FIGS. 20A-20D, with the extension spring 48 biasing the needle actuator body 40 toward the post-use position. When the needle actuator body 40 reaches the post-use position, the lockout arms 78 of actuator button 26 deflect inwards and engage a portion of the needle actuator body 40, thereby preventing further movement of the actuator button 26.

Beneficially, embodiments of the disclosure thus provide a drug delivery system that includes a needle actuator assembly and associated activation mechanism that provide for improved operation of the injector. An actuator button of the activation mechanism is actuated via a simple axial sliding thereof, with no rotation or tilting of the actuator button required to activate the needle actuator assembly. Sliding of the actuator button causes a vertical movement of an activation wedge in the injector, which in turn releases the needle actuator to provide for transitioning of the injector from a pre-use configuration to a use configuration and for actuation of the needle from a retracted position to an extended position. The axial sliding of the actuator button and vertical movement of the activation wedge reduce the likelihood of premature actuation of the injector during a drop impact, which in-turn also prevents an associated premature movement of the needle from its retracted position to its extended position.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. A drug delivery system comprising:
a housing;
a needle actuator assembly comprising:
a needle having a retracted position where the needle is positioned within the housing and an extended position where a portion of the needle protrudes from the housing;
a needle actuator body movable within the housing and including a stop wedge, the needle actuator body movable from a pre-use position where the needle is in the retracted position to a use position where the needle is in the extended position; and
a needle shuttle that secures the needle and is received within the needle actuator body, the needle shuttle configured to engage the needle actuator body as it moves from the pre-use position to the use position to move the needle from the retracted position and the extended position; and
an activation mechanism that interacts with the needle actuator assembly, the activation mechanism comprising:
an actuator button comprising a user interface portion and a button wedge, the actuator button configured to slide axially relative to the housing from a first position to a second position; and
an activation wedge affixed to the housing and comprising a first engagement member engageable with the stop wedge and a second engagement member engageable with the button wedge, with the first engagement member engaged with the stop wedge when the actuator button is in the first position to retain the needle actuator body in the pre-use position;
wherein as the actuator button is slid from the first position toward the second position, the button wedge engages the second engagement member, forcing the activation wedge to move and the first engagement member to become disengaged from the stop wedge, thereby releasing the needle actuator body such that the needle actuator body moves from the pre-use position to the use position and the needle moves from the retracted position to the extended position.

2. The drug delivery system of claim 1, further comprising:
a container received within the housing, the container configured to receive a medicament, the container comprising a stopper configured to move within the container;
a valve assembly configured to pierce a closure of the container and place the medicament within the container in fluid communication with the needle; and
a drive assembly configured to move the container toward the valve assembly and to move the stopper within the container to deliver the medicament to the needle;
wherein the container is spaced from the valve assembly when the actuator button is in the first position, and wherein the container is engaged with the valve assembly when the actuator button is moved toward the second position.

3. The drug delivery system of claim 2, wherein the button wedge comprises a sloped bottom surface and the second engagement member comprises a sloped top surface, and wherein as the actuator button is slid from the first position toward the second position, the sloped bottom surface slides along the sloped top surface and forces the activation wedge to move.

4. The drug delivery system of claim 3, wherein the activation wedge comprises a support leg joined to the housing at one end thereof and to a main body of the activation wedge at another end thereof, the first engagement member provided on the main body and the second engagement member joined to the main body; and
wherein the support leg comprises a spring-type member that actuates between a relaxed configuration and a compressed configuration to vary a length of the support leg.

5. The drug delivery system of claim 4, wherein sliding of the actuator button from the first position toward the second position applies a downward directed force to the activation wedge, causing the support leg to transition from the relaxed configuration to the compressed configuration, and thereby moving the first engagement member and the second engagement member vertically downward.

6. The drug delivery system of claim 5, wherein the first engagement member comprises a vertically-oriented, rearward-facing surface and the stop wedge comprises a vertically-oriented, forward-facing surface, with the rearward-facing surface sliding downward and out of engagement with the forward-facing surface as the support leg transition to the compressed configuration, such that the first engagement member becomes disengaged from the stop wedge.

7. The drug delivery system of claim 6, wherein a vertical distance by which the support leg compresses is greater than a vertical height of the forward-facing surface of the stop wedge.

8. The drug delivery system of claim 4, wherein the stop wedge protrudes radially outward from a remainder of the needle actuator body and the first engagement member protrudes radially inward from a remainder of the main body of the activation wedge.

9. The drug delivery system of claim 1, wherein the needle actuator body comprises guide surfaces formed on a radially inward-facing surface thereof and the needle shuttle comprises cam surfaces thereon, with the needle shuttle and the needle configured to move between the retracted position and the extended position through engagement between the guide surfaces and the cam surfaces.

10. The drug delivery system of claim 1, further comprising an extension spring that biases the needle actuator body from the pre-use position to the use position.

11. The drug delivery system of claim 1, wherein in sliding axially relative to the housing, the actuator button moves only in a horizontal direction, without any tilting of the actuator button.

12. The drug delivery system of claim 1, wherein the needle actuator body is movable to a post-use position, and wherein the actuator button comprises lockout arms that engage a portion of the needle actuator body as the needle actuator body moves to the post-use position, thereby preventing further movement of the actuator button.

13. The drug delivery system of claim 12, wherein the actuator button comprises a central arm and retention arms that engage the housing and the needle actuator body, respectively, during movement of the actuator button between the first position and the second position and during movement of the needle actuator body from the pre-use position, to the use position, and to the post-use position.

14. The drug delivery system of claim 12, further comprising a pad configured to seal the needle, wherein the pad is biased into the needle as the needle actuator body moves from the use position to the post-use position.

15. The drug delivery system of claim 1, wherein the drug delivery system comprises a wearable injector.

16. A drug delivery system comprising:
a housing;
a needle actuator assembly comprising:
a needle having a retracted position where the needle is positioned within the housing and an extended position where a portion of the needle protrudes from the housing; and
a needle actuator body movable within the housing and being movable from a pre-use position where the needle is in the retracted position to a use position where the needle is in the extended position; and
an activation mechanism that interacts with the needle actuator assembly, the activation mechanism comprising:
an actuator button configured to slide axially relative to the housing from a first position to a second position; and
an activation wedge affixed to the housing and comprising a spring-type support leg actuatable between a relaxed configuration and a compressed configuration, with the activation wedge engaged with the needle actuator body when the support leg is in the relaxed configuration and disengaged from the needle actuator body when the support leg is in the compressed configuration;
wherein as the actuator button is slid axially from the first position toward the second position, the actuator button engages the activation wedge and actuates the spring-type support leg from the relaxed configuration to the compressed configuration, such that the activation wedge vertically disengages the needle actuator body, allowing the needle actuator body to move from the pre-use position to the use position.

17. The drug delivery system of claim 16, wherein the needle actuator body comprises a stop wedge, the actuator button comprises a button wedge, and the activation wedge comprises a first engagement member engageable with the stop wedge and a second engagement member engageable with the button wedge, with the first engagement member engaged with the stop wedge when the actuator button is in the first position to retain the needle actuator body in the pre-use position; and
wherein as the actuator button is slid from the first position toward the second position, the button wedge engages the second engagement member, forcing the activation wedge to move and the first engagement member to become disengaged from the stop wedge, thereby releasing the needle actuator body such that the needle actuator body moves from the pre-use position to the use position and the needle moves from the retracted position to the extended position.

18. The drug delivery system of claim 17, wherein the button wedge comprises a sloped bottom surface and the second engagement member comprises a sloped top surface, and wherein as the actuator button is slid from the first position toward the second position, the sloped bottom surface slides along the sloped top surface and applies a downward force to the activation wedge that actuates the spring-type support leg from the relaxed configuration to the compressed configuration, such that the activation wedge is moved downward and vertically disengages the needle actuator body.

19. The drug delivery system of claim 16, further comprising an extension spring that biases the needle actuator body from the pre-use position to the use position.

20. The drug delivery system of claim 16, wherein the needle actuator assembly comprises a needle shuttle that secures the needle and is received within the needle actuator body, the needle shuttle configured to engage the needle actuator body as it moves from the pre-use position to the use position to move the needle from the retracted position and the extended position.
